(19) United States
(12) Patent Application Publication
(10) Pub. No.: US 2014/0234988 A1
(43) Pub. Date: Aug. 21, 2014

(54) Methods for Diagnosing and Treating Medical Conditions Associated With Oxidative Stress

(71) Applicants: Carolin Lackner, Kumberg (AT); Karl Oettl, Graz (AT); Rudolf Stauber, Kumberg (AT); Ruth Birner-Gruenberger, Hitzendorf (AT)

(72) Inventors: Carolin Lackner, Kumberg (AT); Karl Oettl, Graz (AT); Rudolf Stauber, Kumberg (AT); Ruth Birner-Gruenberger, Hitzendorf (AT)

(73) Assignee: MEDIZINISCHE UNIVERSITAET GRAZ, Graz (AT)

(21) Appl. No.: 14/239,532

(22) PCT Filed: Aug. 14, 2012
(86) PCT No.: PCT/EP2012/065913

§ 371 Date: Apr. 29, 2014

(30) Foreign Application Priority Data
Aug. 18, 2011 (EP) 11178007.8

(51) Int. Cl. G01N 33/68

(52) U.S. Cl. CPC G01N 33/6893; G01N 2800/7009
USPC 436/501; 530/389.3; 73/61.43

(57) ABSTRACT

Non-mercaptalbumin-2 is used as a biomarker for diagnosing a medical condition associated with oxidative stress. A method for diagnosing a condition associated with oxidative stress includes determining the redox state of albumin at the cysteine residue at sequence position 34; quantifying the level of non-mercaptalbumin-2 present, non-mercaptalbumin-2 representing the albumin fraction irreversibly oxidized at cysteine 34; and comparing the result to a control level, wherein an elevated level of non-mercaptalbumin-2 as compared to the control level is indicative of the condition associated with oxidative stress. A method for the prevention and/or treatment of a medical condition associated with oxidative stress includes quantifying a level of non-mercaptalbumin-2 that is elevated as compared to a control, wherein such elevated level of non-mercaptalbumin-2 is indicative of the condition associated with oxidative stress; and restoring a control level of albumin by removing excess nonmercaptalbumin-2 and/or exogenously adding albumin.

### CROSS-REFERENCE TO RELATED APPLICATION

This application claims the benefit of the filing date of European Patent Application 11178007.8.6 filed Aug. 18, 2011, the disclosure of which is incorporated herein by reference.

### TECHNICAL FIELD

The present invention relates to methods for the diagnosis and the prevention or treatment of medical conditions associated with oxidative stress, in particular liver diseases, the methods being based on the determination and quantification of the fraction of albumin that is irreversibly oxidized at cysteine 34.

### BACKGROUND

Albumin, a 66.5 kDa protein is the most abundant plasma protein, the main determinant of colloid osmotic pressure and an important carrier for endogenous and exogenous substances (Rothschild, M. A. et al. (1988) Hepatology 8, 385-401). Among its most important functions are fatty acid transport (on several binding areas), drug binding and transport, modulation of capillary permeability, metal chelation, and free radical scavenging which is mediated by the anti-oxidant properties of the sulfur moiety at cysteine 34 (i.e. the cysteine residue at sequence position 34) (reviewed in Evans, T. W. (2002) Aliment Pharmacol. Ther. 16, Suppl. 5, 6-11). Cysteine 34 is the only (of a total of 35) cysteine residue not involved in a disulfide bond. Furthermore, albumin can function as an important buffer for nitric oxide (NO) which is produced in excess in various disease conditions including inflammation circulatory failure, and liver failure. Oxidative damage of albumin at cysteine 34 may impair this NO buffering function and thus contribute to the pathogenesis of the above conditions.

With respect to its antioxidant function, albumin is the major extracellular source of reduced sulfhydryl groups, which are potent scavengers of reactive oxygen and nitrogen species (Quinlan, G. J. et al. (1998) Clin. Sci. 95, 459-465). Depending on the redox state at cysteine 34, there are three major species of human albumin: human mercaptalbumin (HMA), the non-oxidized form with a free thiol group on cysteine 34, and two oxidized species, human nonmercaptalbumin-1 (HNA1) with cysteine, homocysteine or glutathione bound by a disulfide bond and human nonmercaptalbumin-2 (HNA2) with cysteine irreversibly oxidized to sulfinic or sulfonic acid (Sogami, M. et al. (1984) Int. J. Pept. Protein Res. 24, 96-103; Hughes, W. L. and Dintzis, H. M. (1964) J. Biol. Chem. 239, 845-849).

Oxidative stress is believed to play a major role in the pathogenesis of various diseases, such as liver diseases, renal diseases, sepsis, and cancer. This feature may, among other mechanisms, be accomplished via the oxidative modification of albumin. Decreased HMA and/or elevated HNA levels have been reported in chronic liver failure and correlated with severity of the disease (Sogami, M. et al. (1985) J. Chromatogr. 332, 19-27; Oettl, K. et al. (2008) Biochim. Biophys. Acta 1782, 469-473).

However, no robust biomarkers are currently available that enable a reliable and rapid diagnosis of such medical condition and/or an assessment of disease progression. For example, the model for end-stage liver disease (MELD) is a scoring system for assessing the severity of chronic liver disease (Pugh, R. N. et al. (1973) Br. J. Surg. 60, 646-649; Kamath, P. S. et al. (2001) Hepatology 33, 464-470). It uses three different parameters, serum bilirubin, serum creatinine, and the international normalized ratio for prothrombin time (INR, a measure for blood coagulation) to predict survival, and thus are rather laborious and time consuming.

Albumin harbors two specific drug binding sites: site I, which binds large heterocyclic compounds and dicarboxylic acids (such as bilirubin), and site II, which binds aromatic carboxylic compounds (such as benzodiazepines) (Sudlow, G. et al. (1975) Mol. Pharmacol. 11, 824-832). Oxidized albumin shows altered binding capacities for several substances including dansylsarcosine, a model ligand for binding site II (Oettl, K. and Stauber, R. E. (2007) Br. J. Pharmacol. 151, 580-590).

Reduced serum albumin levels are a hallmark of severe liver disease as well as other medical conditions associated with oxidative stress. Besides, several disturbances of albumin binding function are known to occur. However, the pathogenesis of this impaired binding capacity and, specifically, its relation to oxidative albumin damage remains unknown. The lack of suitable biomarkers makes it difficult to determine the prognostic significance of any altered parameters observed on monitoring of disease progression, staging and/or surveillance.

Therefore, the discovery of new biomarkers for this type of medical conditions would be of utmost clinical importance, particularly if these biomarkers would enable a diagnosis and/or prognosis at an early stage of disease progression in order to allow early stage treatment while avoiding unnecessary surgical intervention. Ideally, such new biomarkers would also represent an appropriate target for therapeutic intervention by providing an accurate measure for the severity of a disease.

Accordingly, there still remains a need for improved methods and molecular tools that enable a rapid, reliable and cost-saving diagnosis, staging, monitoring, and therapy of medical conditions associated with oxidative stress.

Hence, it is an object of the present invention to provide such methods and molecular tools.

### SUMMARY

In a first aspect, the present invention relates to a method for diagnosing in a subject a medical condition associated with oxidative stress, the method comprising:

(a) determining in a test sample derived from the subject the redox state of albumin at the cysteine residue at sequence position 34 (cysteine 34);
(b) quantifying the level of non-mercaptalbumin-2 present in the test sample, nonmercapalbumin-2 representing the albumin fraction being irreversibly oxidized at cysteine 34; and
(c) comparing the result obtained in step (b) to a control level, wherein an elevated level of non-mercaptalbumin-2 in the test sample derived from the subject as compared to the control level is indicative of the presence and/or prognosis of the medical condition associated with oxidative stress.

In preferred embodiments, the medical condition associated with oxidative stress is selected from the group consisting of sepsis, renal diseases, liver diseases, cardiovascular diseases, neurodegenerative diseases, rheumatologic diseases, premalignant and malignant diseases, and aging.

In other preferred embodiments, the test sample is a blood sample, and particularly preferably a plasma sample. Preferably, the subject is a human subject.

In specific embodiments, the method further comprises:

(d) determining in the test sample the binding capacity of albumin for aromatic carboxylic compounds; and
(e) comparing the result obtained in step (d) to a control, wherein a decreased binding capacity of albumin in the test sample as compared to the control is indicative of the presence and/or prognosis of the medical condition associated with oxidative stress.

In specific embodiments, the level of non-mercaptalbumin-2 present in the test sample is quantified by means of a technique being selected from the group consisting of a chromatographic technique and a mass spectrometric technique.

In alternative preferred embodiments, the level of non-mercaptalbumin-2 present in the test sample is quantified by means of an antibody-based technique. The antibody molecule employed in these embodiments may exhibit binding specificity for non-mercaptalbumin-2 comprising cysteine 34 in irreversibly oxidized form and binds to the target with an affinity of less than 1 μM, and in particular of less than 100 nM. Preferably, the antibody molecule employed may exhibit binding specificity for an epitope comprising cysteine 34 in irreversibly oxidized form.

In a second aspect, the present invention relates to an antibody molecule exhibiting binding specificity for non-mercaptalbumin-2 comprising cysteine 34 in irreversibly oxidized form and binding to the target with an affinity of less than 1 μM, and in particular of less than 100 nM. Preferably, the antibody molecule exhibits binding specificity for an epitope comprising cysteine 34 in irreversibly oxidized form.

In a third aspect, the present invention relates to a method for the prevention and/or treatment in a subject of a medical condition associated with oxidative stress, the method comprising:

(a) quantifying in a test sample derived from the subject a level of non-mercaptalbumin-2 that is elevated as compared to a control by using a method as defined herein, wherein such elevated level of non-mercaptalbumin-2 in the test sample is indicative of the presence and/or prognosis of the medical condition associated with oxidative stress; and
(b) restoring a control level of albumin by performing either one or both selected from the group consisting of specifically removing excess non-mercaptalbumin-2 and exogenously adding albumin.

In preferred embodiments, the method is performed as an in vitro method.

In a fourth aspect, the present invention relates to the use of non-mercaptalbumin-2 as a biomarker for diagnosing in a subject a medical condition associated with oxidative stress. Preferably, the medical condition associated with oxidative stress to be diagnosed is selected from the group consisting of sepsis, renal diseases, liver diseases, cardiovascular diseases, neurodegenerative diseases, rheumatologic diseases, premalignant and malignant diseases, and aging.

Other embodiments of the present invention will become apparent from the detailed description hereinafter.

### DESCRIPTION OF THE DRAWINGS

FIG. 1: Structure of human albumin.

Shown is the structure of human albumin with different binding sites: site I, which binds large heterocyclic compounds and dicarboxylic acids (such as bilirubin), and site II, which binds aromatic carboxylic compounds (such as benzodiazepines), as determined by Sudlow, G. et al. (1975) Mol. Pharmacol. 11, 824-832. Here, bilirubin is bound covalently to an alternative binding site (lysine 190). Cysteine 34, the only of 35 cystein residues not involved in intramolecular disulphide bonds, is indicated by an arrow. The figure was created using a PDB-file by Zunszain et al. (J. Mol. Biol. (2008) 381, 394-406; PDB-10: 2VUE).

FIG. 2: Determinations of albumin fractions.

In blood samples derived from the study population (9 consecutive patients with acute-on-chronic liver failure (ACLF) being evaluated for extracorporeal liver support and 20 patients with cirrhosis who were candidates for liver transplantation), albumin was fractionated by HPLC as previously described (Kawai, K. et al. (2001) Tokai J. Exp. Clin. Med. 26, 93-99) resulting in three protein fractions representing cysteine 34 as free sulfhydryl form (HMA), as mixed disulfide (HNA1), or in a higher oxidation state (HNA2). The binding properties of the samples were analyzed by using dansylsarcosine as model ligand for binding site II and expressed as IC₅₀ values (i.e. a measure for the concentration of albumin required for the binding of 50% of the ligand present). Shown are boxplots of IC₅₀ values (A) and % HNA2 (B) in different patient groups and controls.

FIG. 3: Analysis of HNA2 by mass spectrometry.

Shown are exemplary results of a mass spectrometry (MS) analysis in a plasma sample of a patient with ACLF in order to determine the oxidative modification of HNA2. In brief, trypsindigested plasma protein was acidified by adding TFA and separated by HPLC as described in Example 1.4. The MS data were analyzed by searching the human SWISSPROT database using Mascot 2.2 software (Matrix Science, London, Great Britain). Human serum albumin was identified with a score of 8636 and amino acid sequence coverage of 93% of the precursor. Oxidation of cysteine 34 to sulfonic acid was detected in the tryptic peptide ALVLIAFAQYLQQCPFEDHVK (cysteine 34 shown in bold) with an ion score of 108 and an expect of 1.1 e-07.

FIG. 4: Correlation of dansylsarcosine binding with clinical chemical parameters and oxidized albumin.

Shown are bivariate correlations of dansylsarcosine binding with INR (prothrombin time, international normalized ratio) (A), bilirubin (B), and % HNA2 (C), respectively. “•” indicates patients with liver failure (ACLF+cirrhosis); “O” indicates patients with sepsis. The lines represent significant correlations within the liver failure group (r>0.5, p<0.05).

FIG. 5: Correlation of dansylsarcosine binding with liver function and inflammation parameters.

Shown are bivariate correlations of dansylsarcosine binding with liver function as reflected by MELD (model for end-stage liver disease) (A and B) and inflammation as reflected by CRP (C-reactive protein) (C and D) within the patients with liver failure (ACLF+cirrhosis). The lines represent significant correlations (r>0.5, p<0.05).

FIG. 6: Diagnostic Accuracy of HNA2 and MELD.

Shown are receiver operating characteristic (ROC) curves of HNA2 and MELD for the prediction of 30 d (A) and 90 d (B) survival.

FIG. 7: Therapeutic concept for the treatment of liver diseases.

Shown is a schematic illustration of an exemplary extracorporeal liver support system that can be employed when performing the methods of the present invention. The liver support system is based on the principle of fractionated plasma separation and adsorption. A plasma fraction of a human subject including nonmercaptalbumin-2 (HNA2) passes an albuminpermeable filter into a secondary circuit where HNA2 is removed by adsorbers containing moieties capable of binding it, such as anti-HNA2 antibodies. After this extracorporeal treatment, fresh albumin is infused to restore the patient's albumin level to normal.

### DETAILED DESCRIPTION

The present invention is based on the unexpected finding that the irreversible oxidation of albumin, that is, the level of non-mercaptalbumin-2 encompassing cysteine 34 irreversibly oxidized to sulfinic acid or sulfonic acid being present in a test sample derived from a subject, is associated with prognosis for medical conditions associated with oxidative stress and thus can be exploited as a new biomarker for the diagnosis, staging, and monitoring of such conditions. With respect to chronic liver disease, the prognostic value of nonmercaptalbumin-2 per se revealed superior diagnostic accuracy as compared to the standard marker MELD, which is based on a combination of three different parameters. Furthermore, the specific removal of excess non-mercaptalbumin-2 being diagnosed in a medical condition associated with oxidative stress represents a new therapeutic approach for the prevention and/or treatment of such conditions.

The present invention illustratively described in the following may suitably be practiced in the absence of any element or elements, limitation or limitations, not specifically disclosed herein.

Where the term “comprising” is used in the description and the claims, it does not exclude other elements or steps. For the purposes of the present invention, the term “consisting of” is considered to be a preferred embodiment of the term “comprising”. If hereinafter a group is defined to comprise at least a certain number of embodiments, this is also to be understood to disclose a group, which preferably consists only of these embodiments.

Where an indefinite or definite article is used when referring to a singular noun, e.g., “a”, an or “the”, this includes a plural of that noun unless specifically stated otherwise.

In case, numerical values are indicated in the context of the present invention the skilled person will understand that the technical effect of the feature in question is ensured within an interval of accuracy, which typically encompasses a deviation of the numerical value given of ±10%, and preferably of ±5%.

Furthermore, the terms first, second, third, (a), (b), (c), and the like, in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

Further definitions of term will be given in the following in the context of which the terms are used. The following terms or definitions are provided solely to aid in the understanding of the invention. These definitions should not be construed to have a scope less than understood by a person of ordinary skill in the art.

In a first aspect, the present invention relates to a method for diagnosing in a subject a medical condition associated with oxidative stress, the method comprising:
(a) determining in a test sample derived from the subject the redox state of albumin at the cysteine residue at sequence position 34 (cysteine 34);
(b) quantifying the level of non-mercaptalbumin-2 present in the test sample, nonmercaptalbumin-2 representing the albumin fraction being irreversibly oxidized at cysteine 34; and
(c) comparing the result obtained in step (b) to a control level, wherein an elevated level of nonmercaptalbumin-2 in the test sample derived from the subject as compared to the control level is indicative of the presence and/or prognosis of the medical condition associated with oxidative stress.

Within the present invention, the terms “diagnosing” (as well as “monitoring”) is intended to encompass not only the mere detection of the presence of a medical condition associated with oxidative stress but also predictions and likelihood analysis. The methods of the present invention are intended to be used clinically in making decisions concerning treatment modalities, including therapeutic intervention, disease staging, and disease monitoring and surveillance. According to the present invention, an intermediate result for examining the condition of a subject may be provided. Such intermediate result may be combined with additional information to assist a physician, nurse, or other practitioner to diagnose that a subject suffers from such a disease or medical condition. Alternatively, the present invention may be used to detect a medical condition in a subject-derived sample, and provide a doctor with useful information to diagnose that the subject suffers from the disease.

Typically, the method of the present invention for diagnosing a medical condition associated with oxidative stress is performed as an in vitro method.

A subject to be diagnosed by the present method is a mammal such as a mouse, rat, hamster, rabbit, cat, dog, pig, cow, horse or monkey. Preferably, the subject to be diagnosed is a human.

The test samples to be employed in the present invention are derived (i.e. collected) from the subject to be diagnosed. The test samples may include body tissues (e.g., biopsies or resections) and fluids, such as blood, sputum, cerebrospinal fluid, and urine. Furthermore, the test samples may contain a single cell, a cell population (i.e. two or more cells) or a cell extract derived from a body tissue. The test samples used in the method of the present invention should generally be collected in a clinically acceptable manner, preferably in a way that nucleic acids and/or proteins are preserved. The test samples may be used in unpurified form or subjected to any enrichment or purification step(s) prior to use, for example in order to isolate the protein fraction comprised in a given sample. The skilled person is well aware of various such purification methods (see, e.g., Sambrook, J., and Russel, D. W. (2001), Molecular cloning: A laboratory manual (3rd Ed.) Cold Spring Harbor, N.Y., Cold Spring Harbor Laboratory Press; Ausubel, F. M. et al. (2001) Current Protocols in Molecular Biology, Wiley & Sons, Hoboken, N.J., USA).

In preferred embodiments, the test sample is a blood sample such as whole blood, plasma, and serum. The term “whole blood”, as used herein, refers to blood with all its constituents (i.e. both blood cells and plasma). The term “plasma”, as used herein, denotes the blood's liquid medium. The term “serum”, as used herein, refers to plasma from which the clotting proteins have been removed. In preferred embodiments, the test sample employed is a plasma sample or a serum sample.

The term “medical condition associated with oxidative stress”, as used herein, relates to any medical condition that is physiologically characterized by the occurrence of oxidative stress. Oxidative stress represents an imbalance between the production and manifestation of reactive oxygen species (such as oxygen radicals and peroxides) and a biological system's ability to readily detoxify the reactive intermediates or to repair the resulting damage (e.g., by synthesis of anti-oxidants such as glutathione). Disturbances in the normal redox state (i.e. reduction-oxidation state) of tissues can cause toxic effects that damage cellular components including proteins, lipids, and nucleic acids.

In preferred embodiments, the medical condition associated with oxidative stress is selected from the group consisting of sepsis, renal diseases, liver diseases, cardiovascular diseases, neurodegenerative diseases, rheumatologic diseases, premalignant and malignant diseases, and aging.

The term “sepsis” (also referred to as “systemic inflammatory response syndrome”), as used herein, denotes a medical condition that is characterized by a systemic inflammatory response affecting the whole body of a subject and the presence of a known or suspected infection by a pathogen such as bacteria, viruses or fungi. In severe cases, sepsis results in organ dysfunction, trouble breathing or blood abnormalities.

The term “renal diseases”, as used herein, refers any disorders or diseases affecting the kidneys including inter alia nephritis, polycycstic kidney disease and acute and chronic renal failure, with chronic renal failure being particularly preferred.

The term “liver diseases”, as used herein, refers to any disorders or diseases affecting the liver including inter alia hepatitis, cirrhosis and acute or chronic liver failure (including acute-on-chronic liver failure), with chronic liver failure being particularly preferred.

The term “cardiovascular diseases”, as used herein, refers to any disorder of the heart and the coronary blood vessels. Examples of cardiovascular diseases include inter alia coronary heart disease, angina pectoris, arteriosclerosis, cardiomyopathies, myocardial infarction, ischemia, and myocarditis.

The term “neurodegenerative diseases”, as used herein, refers to any disorder of the nervous system, in particular of the central nervous system (CNS) (i.e. brain and spinal cord), that are characterized by the progressive loss of structure or function of neurons, including death of neurons. Examples of neurodegenerative diseases include inter alia Alzheimer's disease, Parkinson's disease, and Huntington's disease.

The term “rheumatologic diseases”, as used herein, refers to any disorder affecting the locomotor system including joints, muscles, connective tissues, soft tissues around the joints and bones. Typically, such diseases are associated with inflammation. Examples of rheumatologic diseases include inter alia rheumatoid arthritis, ankylosing spondylitis, gout and systemic lupus erythematosus.

The term “malignant diseases”, as used herein, denotes any type or form of malignant neoplasm (herein also referred to as “cancer”) characterized by uncontrolled division of target cells based on genetic re-programming and by the ability of the target cells to spread, either by direct growth into adjacent tissue through invasion, or by implantation into distant sites by metastasis (where cancer cells are transported through the bloodstream or lymphatic system). Examples include inter alia breast cancer, colorectal cancer, prostate cancer, neuroblastoma, glioblastoma, melanoma, liver cancer, renal cancer, leukemia, lymphomas, and lung cancer. The term “premalignant diseases”, as used herein, denotes any pre-stages of malignant diseases such as benign neoplasms (such as adenomas) or dysplastic lumps or blastomas.

The term “aging”, as used herein, denotes the multidimensional changes in a subject over time, a process also referred to as senescence. It is characterized inter alia by a limited ability of the cells to divide, declining capability to respond to stress, increasing homeostatic imbalance, an increased risk of disease, and the like.

In a first step of the method, the redox state of albumin at the cysteine residue at sequence position 34 (herein referred to as “cysteine 34”) is determined in a test sample derived from the subject to be diagnosed. The term “redox state”, as used herein, refers to the oxidation number of the sulfur moiety at cysteine 34. The oxidation number denotes the charge of the central sulfur atom that it has if all ligands (except carbon) were removed along with the electron pairs that were shared with the central sulfur atom. Accordingly, there are three major species of albumin: mercaptalbumin, the non-oxidized form with a free thiol group on cysteine 34, the oxidized species nonmercaptalbumin-1 with cysteine, homocysteine or glutathione bound by a disulfide bond and nonmercaptalbumin-2 with cysteine irreversibly oxidized to sulfinic or sulfonic acid (Sogami, M. et al. (1984) supra; Hughes, W. L. and Dintzis, H. M. (1964) supra). The term “determining”, as used herein, is to be understood as providing means for distinguishing and/or separating the different fractions of albumin.

Subsequently, the level (i.e. the amount or concentration) of non-mercaptalbumin-2 present in the test sample is quantified. Typically, in healthy humans, mercaptalbumin (HMA) accounts for 70-80%, nonmercaptalbumin-1 (HNA1) for 20-30%, and nonmercaptalbumin-2 (HNA2) for 2-5% of total albumin.

Quantification may be accomplished by any method for determining the concentration of proteins in a sample. Numerous such methods are established in the art including inter alia immunological methods employing antibodies, antibody fragments or antibody-like binding molecules (e.g., enzyme-linked immunosorbent assays), spectrometric methods (e.g., mass spectrometry), and chromatographic methods (e.g., HPLC, high-performance liquid chromatography) (see also, e.g., Sambrook, J., and Russel, D. W. (2001), supra; Ausubel, F. M. et al. (2001) supra). The skilled person is well aware how to perform these methods and how to select a particular method that is appropriate for a given application. In some embodiments, a combination of two or more of the above methods is employed such as a combination of a chromatographic method (e.g., HPLC) and a spectrometric method (e.g., tandem mass spectrometry).

In specific embodiments, the level of non-mercaptalbumin-2 present in the test sample is quantified by means of a chromatographic technique. The term “chromatographic technique”, as used herein, refers to any methods for the separation of mixtures of compounds. The mixture is dissolved in a fluid called the “mobile phase”, which carries it through a structure holding another material called the “stationary phase”. The various constituents of the mixture travel at different speeds, causing them to separate. The separation is based on differential partitioning between the mobile and stationary phases resulting in differential retention of a compound. The mobile phase may be a gas or a liquid or a mixture thereof. In preferred embodiments, liquid chromatography is used for the quantification of the level of non-mercaptalbumin-2, in particular high-performance liquid chromatography, wherein the sample to be separated is forced by a liquid at high pressure (the mobile phase) through a porous stationary phase. Chromatography may be performed inter alfa as affinity chromatography, size-exclusion chromatography or ion exchange chromatography. An exemplary HPLC technique that may be used in the present invention is described in Oettl, K. and Marsche, G. (2010) Meth. Enzymol. 474, 181-195.

In further specific embodiments, the level of non-mercaptalbumin-2 present in the test sample is quantified by means of a spectrometric technique. The term “spectrometric technique”, as used herein, typically refers to any type of mass spectrometry (MS), that is, a method for determining the mass-to-charge ratio of charged compounds or particles. In brief, the (fragmented) components of the sample to be analyzed are ionized, e.g., by impacting them with an electron beam, resulting in the formation of ions. These ions are separated according to their mass-to-charge ratio by means of electromagnetic fields. The ion signal detected is then processed into mass spectra. Multiple rounds of mass spectrometry, commonly separated by any form of molecule fragmentation, can be coupled (tandem-MS). There are various methods for fragmenting molecules for tandem MS, including inter alia collision-induced dissociation (CID), electron capture dissociation (ECD), electron transfer dissociation (ETD), infrared multi-photon dissociation (IRMPD), blackbody infrared radiative dissociation (BIRD), electron-detachment dissociation (EDD) and surface-induced dissociation (SID). All these techniques are well established in the art.

In alternative preferred embodiments, the level of non-mercaptalbumin-2 present in the test sample is quantified by means of an antibody-based (i.e. immunological) technique. Such techniques employ an antibody or an antibody-like molecule which specifically recognizes non-mercaptalbumin-2, that is, which exhibits binding specificity for non-mercaptalbumin-2 (without substantial cross-reactions with either non-mercaptalbumin-1 or mercaptalbumin). The antibody may be a polyclonal or, preferably, a monoclonal antibody. Instead of a full-length antibody molecule, any fragments or variants derived thereof may be employed provided that they retain substantial binding specificity for non-mercaptalbumin-2 (i.e. at least 60%, at least 65%, at least 70%, at least 75%, at least 80%, at least 85%, at least 90%, or at least 95% of the binding specificity of a full-length antibody). Examples of such fragments or variants include inter alia Fab fragments, Fab′ fragments, F(ab′)₂ fragments, single-domain antibodies, single-chain variable fragments, and trifunctional antibodies. Furthermore, antibody mimetics, such as anticalins, affibodies, or monobodies, may be used as well. All these types of molecules as well as their preparation are well known in the art (cf., e.g., Meinders, F. (2001) Current Protocols in Immunology. Wiley & Sons, Hoboken, N.J., USA; Harlow, E. and Lane, D. (2008) Antibodies—A Laboratory Manual. Cold Spring Harbor Laboratory Press, Cold Spring Harbor, N.Y.).

Typically, the antibody molecule employed in these embodiments binds to the target protein (i.e. non-mercaptalbumin-2) with an affinity (i.e. dissociation constant of the complex between antibody and target) of less than 1 μM or less than 800 nM, preferably with an affinity of less than 500 nM or less than 200 nM, and particularly preferably with an affinity of less than 100 nM, such as 90 nM, 80 nM, 70 nM, 60 nM, 50 nM, 40 nM, 30 nM, 20 nM, 15 nM, 10 nM, 8 nM, 5 nM, 2 nM, 1 nM or less than 1 nM. Preferably, the cross-reactivity of the antibody with both non-mercaptalbumin-1 and mercaptalbumin is less than 10% of the total binding activity, more preferably less than 5% and particularly preferably less than 3%_{.}

Preferably, the antibody molecule employed may exhibit binding specificity for an epitope comprising cysteine 34 in irreversibly oxidized form. That is, the antibody recognizes and binds to a sequence region of non-mercaptalbumin-2 that encompasses cysteine 34. The epitope may represent a stretch of consecutive amino acid residues comprising cysteine 34. Alternatively, the epitope recognized by the antibody may be a “conformational” epitope, that is, a particular three-dimensional structure (i.e. conformation or folding of the protein target) being specific for non-mercaptalbumin-2. Such conformational epitope may or may not encompass cysteine 34.

Detection of the antibody bound to non-mercaptalbumin-2 may be performed by any suitable immunological detection protocol available in the art, such as enzyme-linked immunosorbent assay (ELISA), radioimmunoassay, magnetic immunoassay, and the like. Preferably, an ELISA is performed. The skilled person is well aware of all these techniques. For detection, one or more labels may be directly attached to the (primary) antibody recognizing nonmercaptalbumin-2. Alternatively, a labeled secondary antibody which is specific for the primary antibody may be employed. Labels that may be used herein include any compound, which directly or indirectly generates a detectable compound or signal in a chemical, physical or enzymatic reaction. Labeling and subsequent detection can be achieved by methods well known in the art (see, for example, Sambrook, J., and Russel, D. W. (2001), supra; and Lottspeich, F., and Zorbas H. (1998) Bioanalytik, Spektrum Akademischer Verlag, Heidelberg/Berlin, Germany). The labels can be selected inter alia from fluorescent labels, enzyme labels, chromogenic labels, luminescent labels, radioactive labels, haptens, biotin, metal complexes, metals, and colloidal gold. All these types of labels are well established in the art and can be commercially obtained from various suppliers. An example of a physical reaction that is mediated by such labels is the emission of fluorescence or phosphorescence upon irradiation. Alkaline phosphatase, peroxidase, 13-galactosidase, and 13-lactamase are examples of enzyme labels, which catalyze the formation of chromogenic reaction products, and which may be used in the invention.

In a further aspect, the present invention relates to an antibody molecule, as described herein above, that exhibits binding specificity for non-mercaptalbumin-2 comprising cysteine 34 in irreversibly oxidized form and binds to the target with an affinity of less than 1 μM, and in particular of less than 100 nM. Preferably, such antibody molecule exhibits binding specificity for an epitope comprising cysteine 34 in irreversibly oxidized form.

In a final step of the method according to the present invention, the results obtained when analyzing the test sample (that is, the level of non-mercaptalbumin-2) is compared to a control level. The term “control level”, as used herein, relates to a level of nonmercaptalbumin-2 which may be determined at the same time as the test sample by using (a) sample(s) derived from a healthy subject or a subject whose disease state is known. Preferably, the level of non-mercaptalbumin-2 of a healthy subject is used as a control.

Alternatively, the control level may be determined by a statistical method based on the results obtained by analyzing previously determined level(s) of non-mercaptalbumin-2 in samples from subjects whose disease state is known. Furthermore, the control level may be derived from a database from previously tested subjects or cells. Moreover, the level of nonmercaptalbumin-2 in a test sample may be compared to multiple control levels, as determined from multiple reference samples.

The term “elevated level” in the context of the present invention denotes an increase of the amount or concentration of non-mercaptalbumin-2 in the test sample as compared to the control level. Levels are deemed to be “elevated” when the amount in the test sample exceeds the control level by, for example, at least 10%, at least 20%, at least 30%, at least 40%, at least 50%, at least 60%, at least 70%, at least 80%, at least 90%, at least 100%, at least 120%, at least 140%, at least 160%, at least 180%, at least 200%, at least 250%, at least 300%, at least 350%, at least 400%, at least 450%, at least 500%, or more than at least 500%, or when the amount in the test sample is at least 0.1 fold, at least 0.2 fold, at least 0.5 fold, at least 1 fold, at least 2 fold, at least 3 fold, at least 4 fold, at least 5 fold, at least 6 fold, at least 7 fold, at least 8 fold, at least 9 fold or at least 10 fold or more in comparison to the control level. Such an elevated level is indicative for the presence and/or the prognosis (i.e. predictions on disease progression) of the medical condition associated with oxidative stress. Typically, the difference between the level in the test sample and the control is indicative of the severity of the medical condition, that is, a rather small difference (e.g., 10% or 50%) likely indicates a non-severe form of the condition, while a big difference (e.g. 300% or 400%) points to an advanced or even end-stage condition.

In specific embodiments, the method further comprises the quantification of the level of nonmercaptalbumin-1 and/or of the level of mercaptalbumin in the test sample to be analyzed and a comparison of the values obtained with respective control levels. Typically, an elevated level of non-mercaptalbumin-1 and/or a decreased (i.e. reduced) level of mercaptalbumin in the test sample as compared to the control level is/are indicative of the presence and/or prognosis of the medical condition associated with oxidative stress.

In specific embodiments, the method further comprises:

(d) determining in the test sample the binding capacity of albumin for aromatic carboxylic compounds; and
(e) comparing the result obtained in step (d) to a control, wherein a decreased binding capacity of albumin in the test sample as compared to the control is indicative of the presence and/or prognosis of the medical condition associated with oxidative stress.

Albumin binds aromatic carboxylic compounds (such as benzodiazepines) via binding site II as defined by Sudlow et al. (Sudlow, G. et al. (1975) supra). Binding capacity of albumin for such compounds may be determined by using any suitable aromatic carboxylic compound as a ligand in binding analysis measuring binding affinity and/or specificity. One exemplary model ligand for this purpose is dansylsarcosine. The assay may be performed as described in the experimental section below. However, the skilled person is well aware of any modifications of this approach or of alternative techniques for performing such ligand binding assays.

As already described for the determination of the level of non-mercaptalbumin-2, the results of the binding analysis of albumin for aromatic carboxylic compounds is compared to a control level, wherein a decreased binding capacity of albumin in the test sample as compared to the control is indicative of the presence and/or prognosis of the medical condition associated with oxidative stress. For example, the binding capacity in the test sample may be reduced by at least 10%, at least 20%, at least 30%, at least 40%, at least 50%, at least 60%, at least 70%, at least 80%, at least 90%, at least 100%, at least 120%, at least 140%, at least 160%, at least 180%, at least 200%, at least 250%, at least 300%, at least 350%, at least 400%, at least 450%, at least 500%, or more than at least 500% as compared to the control level.

In specific embodiments, the method of the invention may further comprise determining in the test sample derived from the subject any one or more of the parameters selected from the group consisting of bilirubin, creatinine, C-reactive protein, and pro-thrombin time (international normalized ratio) and comparing the values obtained with respective control levels.

In a third aspect, the present invention relates to a method for the prevention and/or treatment in a subject of a medical condition associated with oxidative stress, the method comprising:

(a) quantifying in a test sample derived from the subject a level of non-mercapalbumin-2 that is elevated as compared to a control by using a method as defined herein, wherein such elevated level of non-mercapalbumin-2 in the test sample is indicative of the presence and/or prognosis of the medical condition associated with oxidative stress; and
(b) restoring a control level of albumin by performing either one or both selected from the group consisting of specifically removing excess non-mercaptalbumin-2 and exogenously adding albumin.

In preferred embodiments, the method is performed as an in vitro method.

The respective terms are used in accordance with the definitions given herein above.

The method comprises in a first step the “diagnosing” of a medical condition associated with oxidative stress via the quantification of the level of non-mercaptalbumin-2 that is elevated as compared to a control level (e.g., the level typically observed in healthy controls). In other words, information is provided that the subject from whom the test sample is derived suffers from a medical condition associated with oxidative stress. Based on this information, a control level (i.e., for example, a normal amount of a healthy subject) of albumin is restored in the subject to be treated by performing either one or both selected from the group consisting of specifically removing excess non-mercaptalbumin-2 and exogenously adding albumin. The skilled person is well aware how to select a suitable treatment regimen depending on the constitution of the subject to be treated, the medical condition concerned, the amount of non-mercaptoalbumin-2 present, and the like.

In some embodiments, the method of the invention involves the specific removal of excess non-mercaptalbumin-2 present in the test sample derived from the subject (or in the subject per se, for example, via blood dialysis), for example by means of adsorbing nonmercaptalbumin-2 at a binding matrix (e.g., an antibody molecule, optionally immobilized on a support) or by means of filtration. In other embodiments, the method involves the exogenous addition of albumin to a blood sample derived from the subject (which may then be administered to the subject to be treated), to a blood preservation of a donor subject (which may then be administered to the subject to be treated), or may be directly administered to the subject to be treated, e.g., by infusion or injection. In yet other embodiments, the method involves a combination of removing excess non-mercaptalbumin-2 and the exogenous addition of albumin.

In case of liver diseases, the method of the invention may be performed by using an extracorporeal liver support system. Several such artificial systems are well established in the art, for example, the Molecular Adsorbents Recirculating System (MARS®, Gambro, Lund, Sweden), a variant of albumin dialysis, and Prometheus® (Fresenius Medical Care, Bad Homburg, Germany), a device for fractionated plasma separation via an albuminpermeable filter that was developed to improve removal of albumin-bound toxins (these systems are reviewed, e.g., in Krisper, P. and Stauber, R. E. (2007) Nat. Clin. Pract. Nephrol. 3, 267-276). So far, such systems are typically adapted for removal of albumin-bound toxins but can be readily modified for the removal of a particular fraction of plasma albumin (i.e. non-mercaptalbumin-2). An exemplary embodiment of an extracorporeal liver support system that can be employed when practicing the present invention is shown in FIG. 6.

In a further aspect, the present invention relates to the use of non-mercaptalbumin-2 as a biomarker (i.e. a molecular indicator) for diagnosing in a subject a medical condition associated with oxidative stress. Preferably, the medical condition associated with oxidative stress to be diagnosed is selected from the group consisting of sepsis, renal diseases, liver diseases, cardiovascular diseases, neurodegenerative diseases, cancer, and aging.

The invention is further described by the figures and the following examples, which are solely for the purpose of illustrating specific embodiments of this invention, and are not to be construed as limiting the scope of the invention in any way.

### EXAMPLES

### Example 1

### Patients and Methods

### 1.1 Patients

The study population comprised 9 consecutive patients with acute-on-chronic liver failure (ACLF) being evaluated for extracorporeal liver support as well as 20 patients with cirrhosis who were candidates for liver transplantation. ACLF was defined as acute deterioration of liver function over a period of 2-4 weeks, associated with a precipitating event, with jaundice and either hepatic encephalopathy or hepatorenal syndrome, and with a high SOFA (sepsis related organ failure assessment) or APACHE II (acute physiology and chronic health evaluation II) score (Jalan, R. and Williams, R. (2002) Blood Purif. 20, 252-261). Patients with hepatocellular carcinoma or recent gastrointestinal bleeding (<7 days before enrollment) were excluded. Sepsis was diagnosed according to international guidelines (Bone, R. C. et al. (1992) Chest 101, 1644-1655) as the presence of the systemic inflammatory response syndrome (SIRS) in response to a confirmed infectious process. Systemic Inflammatory response syndrome was defined by the presence of two or more of the following criteria: Temperature >38° C. or <36° C.; heart rate >90 beats/min; respiratory rate >20 per min or PaCO₂<32 mm Hg; white blood cells >12.000 cells/mm³ or <4000 cells/mm³. Infection was defined as positive cultures of blood, ascites, urine, sputum or wounds and/or clinical findings suggestive for infections (chest X-ray).

Patients were managed following local treatment protocols for liver failure and sepsis, including full intensive care support. Patients with suspected hepatorenal syndrome received an intravenous fluid challenge with albumin 1 g/kg according to current guidelines. Plasma samples from 15 age- and sex-matched healthy blood donors were used as controls. The study protocol was approved by the Ethics Committee of the Medical University of Graz (Austria), and informed consent was obtained in accordance with the Declaration of Helsinki.

### 1.2 Study Design

Blood samples were collected within 24 hours after admission, immediately centrifuged at 4° C., and plasma aliquots were stored at −70° C. until batch analysis. Bilirubin, albumin, creatinine, prothrombin time (international normalized ratio, INR), and C-reactive protein (CRP) were routinely assessed. To estimate severity of liver disease, the model for end-stage liver disease (MELD) was calculated (Pugh, R. N. et al. (1973) Br. J. Surg. 60, 646-649; Kamath, P. S. et al. (2001) Hepatology 33, 464-470).

### 1.3 Albumin Analysis

Albumin was fractionated by high performance liquid chromatography (HPLC) to give three peaks representing cysteine 34 in the free sulfhydryl form (HMA; human mercaptalbumin), as a mixed disulfide (HNA1; human non-mercaptalbumin-1) or in a higher oxidation state (HNA2; human non-mercaptalbumin-2) as previously described (Kawai, K. et al. (2001) Tokai J. Exp. Clin. Med. 26, 93-99). Briefly, plasma samples were diluted 1:100 with 0.1 M sodium phosphate, 0.3 M sodium chloride, pH 6.87, filtered through a Whatman 0.45 μm nylon filter (Bartelt Labor- & Datentechnik, Graz, Austria). Thereafter, 20 μl were injected into the HPLC system. Separation was performed using a Shodex Asahipak ES-502N 7C anion exchange column (7.5×100 mm, Bartelt Labor- & Datentechnik, Graz, Austria) with 50 mM sodium acetate, 400 mM sodium sulfate, pH 4.85, as mobile phase. For elution, a gradient of 0% to 6% ethanol and a flow rate of 1 ml/min applied by a FLUX Rheos 4000 gradient pump (Spectronex, Vienna, Austria) were used. The column was kept at 35° C. Detection was carried out by means of determining fluorescence at 280/340 nm with a Jasco 821FP fluorescence detector (Spectronex, Vienna, Austria). Quantification was based on peak heights as determined by EZ Chrom Elite chromatography software (VWR, Vienna, Austria). A commercial 20% albumin solution obtained from Behring GmbH (Vienna, Austria) was used as a control.

1.4 Mass Spectrometry

In order to further determine oxidative modification of HNA2, mass spectrometry was performed in a plasma sample. Disulfide bridges of plasma proteins were reduced by incubation with 5 mM DTT for 20 min under shaking at 550 rpm and 56° C. and then alkylated by incubation with 5 mM iodoacetamide for 15 min under shaking at 550 rpm and room temperature. Subsequently, protein was digested by adding modified trypsin (purchased from Promega; Mannheim, Germany; trypsin to plasma protein 1:50 (w/w)) and shaking over night at 550 rpm and 37° C. The peptide solution was acidified by adding 0.05% trifluoracetic acid (TFA, final concentration) and diluted in solvent A to a theoretical final total peptide concentration of 25 ng/μl.

Digests were separated by nano-HPLC (Dionex UltiMate 3000 RSLC system, Vienna, Austria) equipped with a Zorbax 3005B-C18 enrichment column (5 μm, 5×0.3 mm) and a Zorbax 3005B-C18 nanocolumn (3.5 μm, 150×0.075 mm). 500 ng of sample (20 μl) were injected and concentrated on the enrichment column for 6 min using 0.05% TFA as isocratic solvent at a flow rate of 20 μl/min. The column was switched in the nanoflow circuit and the sample loaded on the nanocolumn at a flow rate of 300 nl/min. Separation was performed using the following gradient, where solvent A is 0.05% TFA in water and solvent B is a mixture of 80% acetonitrile in water containing 0.05% TFA; 0-2 min: 4% B; 2-180 min: 4-28% B; 180-255 min: 28-50% B, 255-260 min: 50-95% B; 260-279 min: 95% B; 279-280 min: 95-4% B; 280-300 min: re-equilibration at 4% B. The sample was ionized in the nanospray source equipped with nanospray tips (PicoTip™, Coating: 1 P-4P, 15±1 μm Emitter, New Objective, Woburn, Mass., USA) and analyzed in a Thermo Orbitrap velos pro mass spectrometer (Thermo Fisher Scientific, Waltham, Mass., USA) operated in positive ion mode, applying alternating full scan MS (m/z 200 to 2000) in the ion cyclotron and MS/MS by collision induced dissociation of the 20 most intense peaks in the ion trap with dynamic exclusion enabled.

The LC-MS/MS data were analyzed by searching the human SWISSPROT database (http://www.uniprot.org/) with Mascot 2.2 (MatrixScience, London, Great Britain). Oxidation to sulfonic acid at Cys (amino acid) residues, phosphorylation on Ser, Thr or Tyr residues, and oxidation on Met residues were included as variable modifications next to carbamidomethylation on Cys residues as fixed modification. No enzyme cleavage specificity was used. A maximum false discovery rate of 5% using decoy database search, an ion score cut off of 20 and a minimum of two identified peptides were chosen as identification criteria.

### 1.5 Binding of Dansylsarcosine

The capacity of the albumin binding site II, which binds aromatic carboxylic compounds such as benzodiazepines (Sudlow, G. et al. (1975) Mol. Pharmacol. 11, 824-832), was determined using dansylsarcosine (OS) as a model ligand and determined by means of HPLC according to the method described (Watanabe, H. et al. (2001) Pharm. Res. 18, 1775-1781). Plasma albumin concentrations were determined by using an automatic clinical chemical analyser (EuroLyser, Eurolyser Diagnostica, Salzburg, Austria).

Serum samples were diluted with PBS and adjusted to an albumin concentration of 40 μM. Based on the 40 μM sample, a dilution series with final concentrations of 20 μM, 10 μM, and 5 μM albumin (in PBS) was made. Each of these samples (250 μl) was mixed with the same volume of 10 μM DS (Sigma-Aldrich, Vienna, Austria) solution. Unbound DS molecules were separated from the plasma components via ultrafiltration (Ultrafree-MC reg. Cellulose 30K, Millipore, Vienna, Austria) at 500 g for 10 min at room temperature. 150 μl of the flow through were put into HPLC vials. As a control, 10 μM DS was diluted with the same volume of Millipore water and ultrafiltered as described. For an external standard curve, a dilution series of DS with Millipore water (10 μM, 5 μM, 2.5 μM, and 1.25 μM) was prepared. 20 μl of the filtrate were then injected to the HPLC system. Separation was performed by means of a Waters-Spherisorb 3 ODS2 column (Waters, Vienna, Austria), using a 1:1 (v/v) mixture of acetonitrile and 100 mM sodium acetate (pH 4.5) as the mobile phase. A flow rate of 500 μl/min was applied by a FLUX Rheos 2000 pump (Spectronex, Vienna, Austria) and used for isocratic elution. The column was kept at a temperature of 25° C. DS was detected by means of determining fluorescence at 482/350 nm using a Hitachi F1050 (Merck) detector. Quantificaton via the peak heights was done using the EZ Chrom Elite chromatography software (VWR, Vienna, Austria). From the straight line obtained by the graph of ln (free DS) vs. albumin, the albumin concentration binding half of the ligand was calculated and given as IC₅₀ (μmol/l). An increase in IC₅₀ therefore indicates an impaired binding capacity of albumin binding site II.

### 1.6 Statistics

Results are given as mean±SD (standard deviation) unless indicated otherwise. For all inferential statistical calculations, SPSS 16.0 (Statistical Package for the Social Sciences, version 16.0) was used. The relationship between the several blood parameters determined is described with Spearman's rank correlation coefficient. Multiple linear regression analysis was used to account for the inter-correlation structure between independent variables and DS binding. Group means were compared by analysis of variance (ANOVA). The diagnostic accuracy of prognostic variables was examined by means of receiver operating characteristic (ROC) analysis.

### Example 2

### Results

The characteristics of the study population are shown in Table 1.

The international normalized ratio (INR) was in the same range in patients with cirrhosis, ACLF or sepsis. As expected, in all three patient groups the plasma levels of CRP, creatinine (except cirrhosis) and bilirubin were significantly increased and albumin significantly decreased compared to controls. Septic patients presented with the lowest albumin, highest CRP and highest creatinine, ACLF patients with the highest bilirubin values. Mean values of leukocytes were above the normal range in septic and ACLF patients.

TABLE 1
Patient characteristics

| | Control | Cirrhosis | ACLF | Sepsis |
|---|---|---|---|---|
| | (n = 15) | (n = 20) | (n = 9) | (N = 18) |
| Age (yrs) | 55 ± 5 | 58 ± 9 | 48 ± 8 | 62 ± 15 |
| Sex (m:f) | 9:6 | 14:6 | 7:2 | 13:5 |
| Etiology: alcohol | n/a | 85% | 100% | n/a |
| Infection ^{a} | n/a | 5% | 78% | 100% |
| Albumin (g/dl) | 4.8 ± 0.2 | 3.3 ± 0.6 | 3.3 ± 0.7 ^{b} | 2.3 ± 0.4 |
| Bilirubin (mg/dl) | 0.6 ± 0.3 | 5.0 ± 7.3 | 23.6 ± 9.0 | 3.6 ± 5.2 |
| INR | n/a | 1.4 ± 0.4 | 2.2 ± 0.5 | 1.4 ± 0.8 |
| Creatinine (mg/dl) | 0.8 ± 0.1 | 1.5 ± 1.4 | 1.7 ± 1.0 | 2.1 ± 2.1 |
| Leukocyte (x 10⁻⁹/l) | n/a | 6.6 ± 3.5 | 14.5 ± 6.4 | 13.5 ± 9.3 |
| CRP (mg/l) | 2 ± 2 | 25 ± 37 | 75 ± 59 | 165 ± 77 |
| MELD ^{c,d} | n/a | 13 (9) | 30 (7) | n/a |
| Hospital mortality | n/a | 10% | 67% | 56% |

INR, international normalized ratio; CRP, C-reactive protein; n/a, not available
^{a} presence of infectious focus and/or positive blood culture
^{b} 5 out of 9 ACLF patients received albumin infusions due to hepatorenal syndrome
^{c} MELD score was obtained using the MELD calculator at the Mayo Clinic website
(http://www.mayoclinic.org/gi-rst/mayomodel5.html)
^{d} Median (IQR)

In all patient groups, oxidized albumin fractions were increased whereas HMA was decreased as compared to controls (Table 2, FIG. 2). HNA2 was increased strikingly in ACLF as compared to cirrhosis and sepsis.

TABLE 2
Albumin species and binding properties in patients and controls

| | Control | Cirrhosis | ACLF | Sepsis |
|---|---|---|---|---|
| HMA (%) | 65 ± 4 | 47 ± 20* | 43 ± 14* | 49 ± 17* |
| HNA1 (%) | 31 ± 4 | 46 ± 18* | 42 ± 15* | 44 ± 17* |
| HNA2 (%) | 4 ± 1 | 8 ± 5* | 15 ± 3* | 8 ± 5* |
| IC₅₀ (g/l) | 12 ± 2 | 13 ± 4 | 25 ± 8* | 18 ± 7* |

*p < 0.05 compared to controls, by ANOVA and post-hoc test

While there was no difference in DS binding between controls and cirrhotic patients, IC₅₀-values were increased in the septic and ACLF group indicating impaired albumin binding capacity in these patients.

Parallel HPLC analysis and mass spectrometry (MS/MS) in a plasma sample of one patient with ACLF in order to determine the oxidative modification of HNA2 revealed the specific oxidation of cysteine 34 to sulfonic acid confirming that the allocated HNA2 peak in the HPLC chromatogram can be attributed to oxidative modification of HNA2 (FIG. 3). The MS data were analyzed by searching the human SWISSPROT database using Mascot 2.2 software (Matrix Science, London, Great Britain). Human serum albumin was identified with a score of 8636 and amino acid sequence coverage of 93% of the precursor. Oxidation of cysteine 34 to sulfonic acid was detected in the tryptic peptide ALVLIAFAQYLQQCPFEDHVK (cysteine 34 shown in bold) with an ion score of 108 and an expect of 1.1 e-07.

In patients with liver disease (ACLF and cirrhosis), the binding of DS was related to INR, bilirubin and HNA2 (Table 3, FIG. 4), and the MELD score (a composite of bilirubin, INR and creatinine and measure for overall disease severity) showed a significant correlation with IC₅₀ (FIG. 5). No significant correlation of IC₅₀ with CRP was found (FIG. 5). Albumin fractions HMA and HNA2 correlated significantly with bilirubin revealing an irreversible oxidation of albumin with increasing bilirubin levels (Table 3). HNA2 correlated significantly with MELD and CRP (FIG. 5). Using a stepwise regression model we found that only bilirubin (p=0.008) and INR (p=0.038) had an independent impact on DS binding, while albumin fractions showed no further significant contribution to impaired DS binding. Within the group of septic patients no correlation with IC₅₀-values was found. Receiver operating characteristic (ROC) analysis revealed superior diagnostic accuracies of HNA2 as compared to MELD as predictors of both 30-day and 90-day survival (Table 4, FIG. 6).

TABLE 3
Significant correlations of total serum albumin fractions and DS
binding capacity in liver failure (ACLF + cirrhosis)

| Variable 1 | Variable 2 | R | P |
|---|---|---|---|
| HMA | Bilirubin | −0.52 | 0.005 |
| HNA2 | CRP | 0.68 | <0.001 |
| | Bilirubin | 0.63 | <0.001 |
| | INR | 0.59 | 0.001 |
| IC₅₀ | INR | 0.82 | <0.001 |
| | Bilirubin | 0.82 | <0.001 |
| | HNA2 | 0.57 | 0.001 |
| | Age | −0.54 | 0.002 |

CRP, C-reactive protein; INR, international normalized ratio. Only correlations demonstrating at least moderate association (R²>0.25) are shown.

[The remainder of this page is intentionally blank.]

TABLE 4
Performance of HNA2 and MELD as predictors of 30-day and
90-day survival in patients with chronic liver failure

| | AUROC | AUROC |
|---|---|---|
| | (30 d survival) | (90 d survival) |
| HNA2 | 0.88 (0.74-1.00) | 0.87 (0.72-1.00) |
| MELD | 0.80 (0.60-0.99) | 0.77 (0.58-0.96) |

AUROC, area under the receiver operating characteristic curve
HNA2, human nonmercaptalbumin-2
MELD, model for end-stage liver disease
Values in parentheses denote 95% confidence intervals

### Example 3

### Discussion

In the present study, marked alterations of the redox state of albumin and a significant impairment of albumin binding capacity (site II) were observed in patients with chronic liver failure as well as in septic patients without liver failure. This is in line with previous finding of decreased binding of bilirubin to oxidized albumin fractions. In the present study, multivariate analysis did not reveal a major correlation of impaired albumin binding capacity for DS to the redox state of albumin. This indicates that impaired albumin binding capacity of site II in liver patients cannot solely be attributed to their altered albumin redox state. Instead, albumin binding capacity was mainly related to markers of liver dysfunction such as INR and bilirubin.

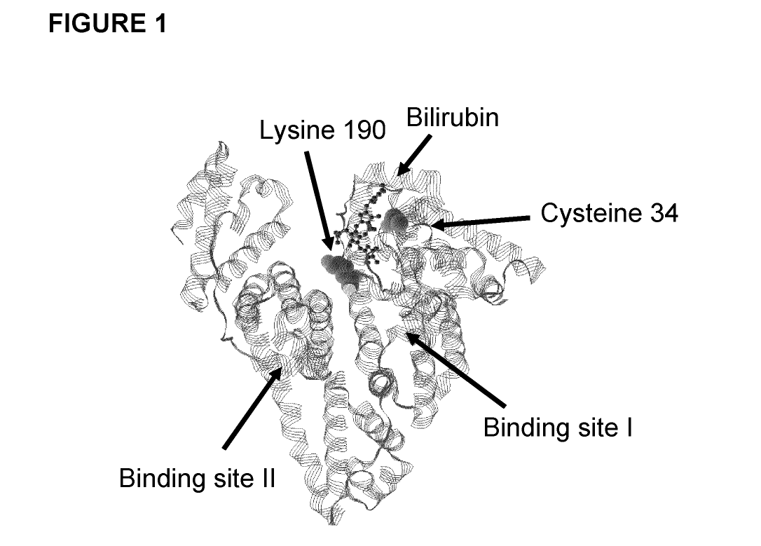

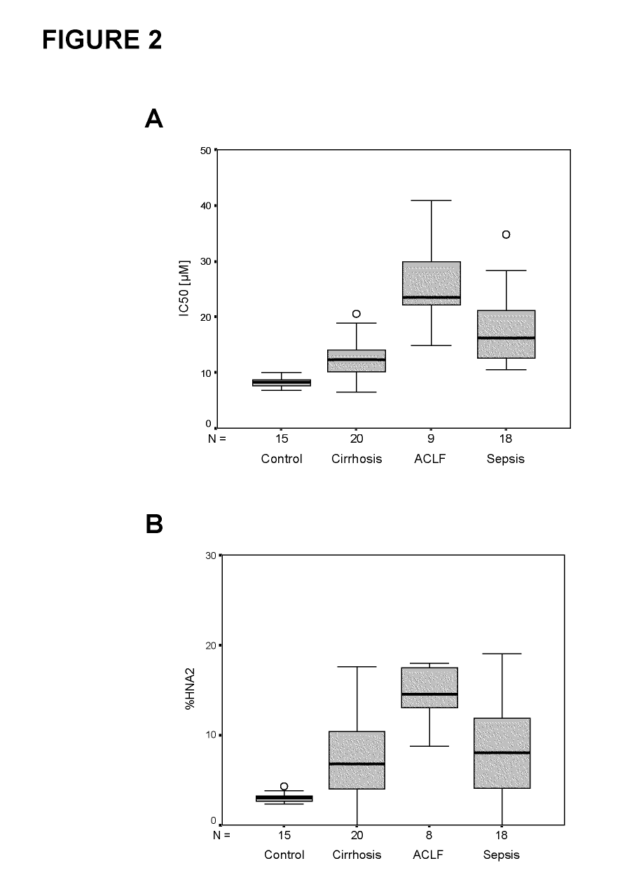

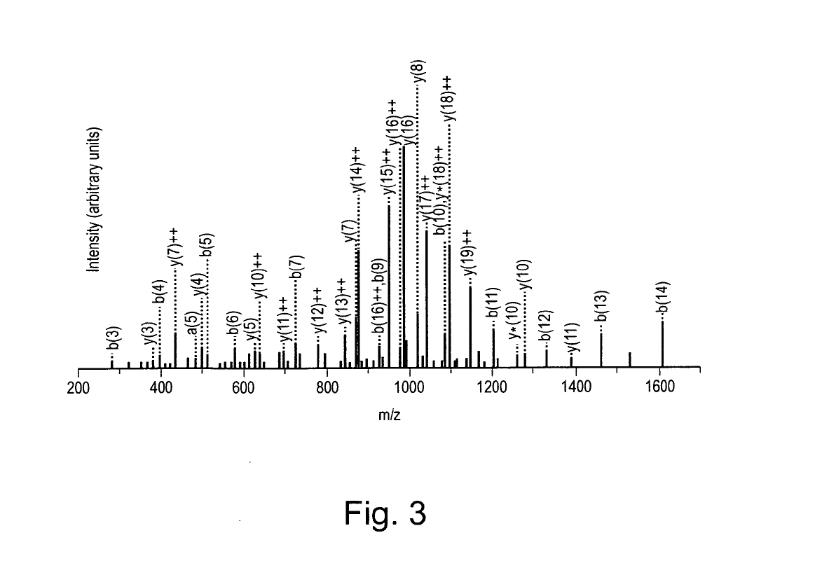

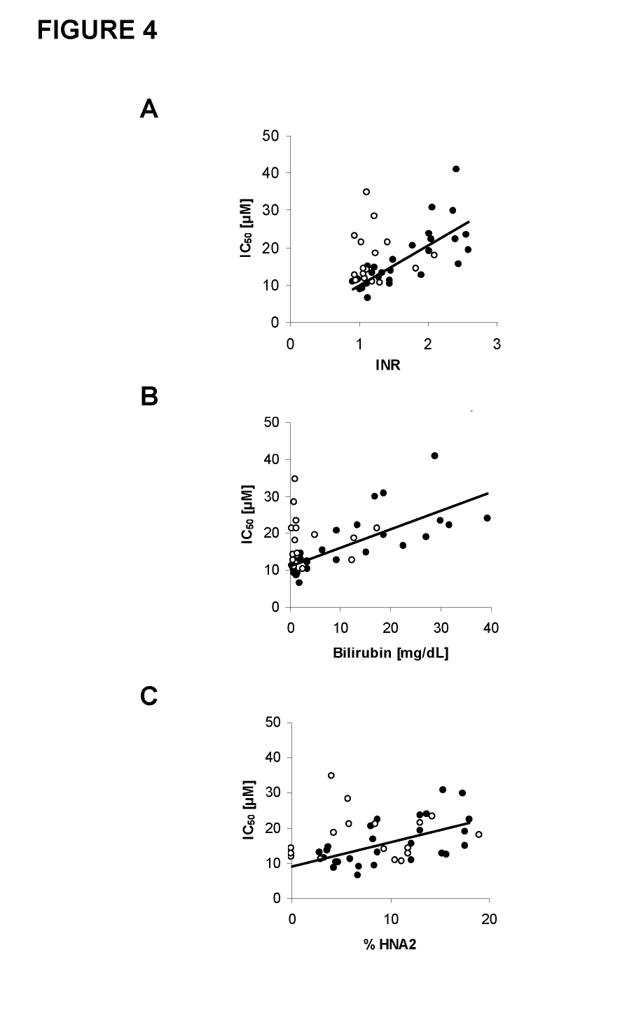

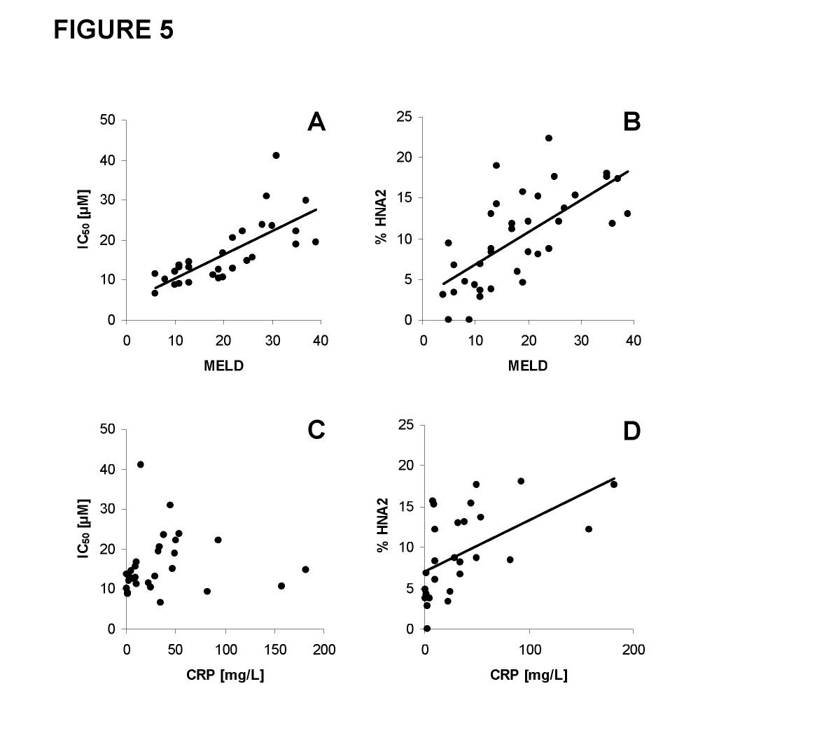

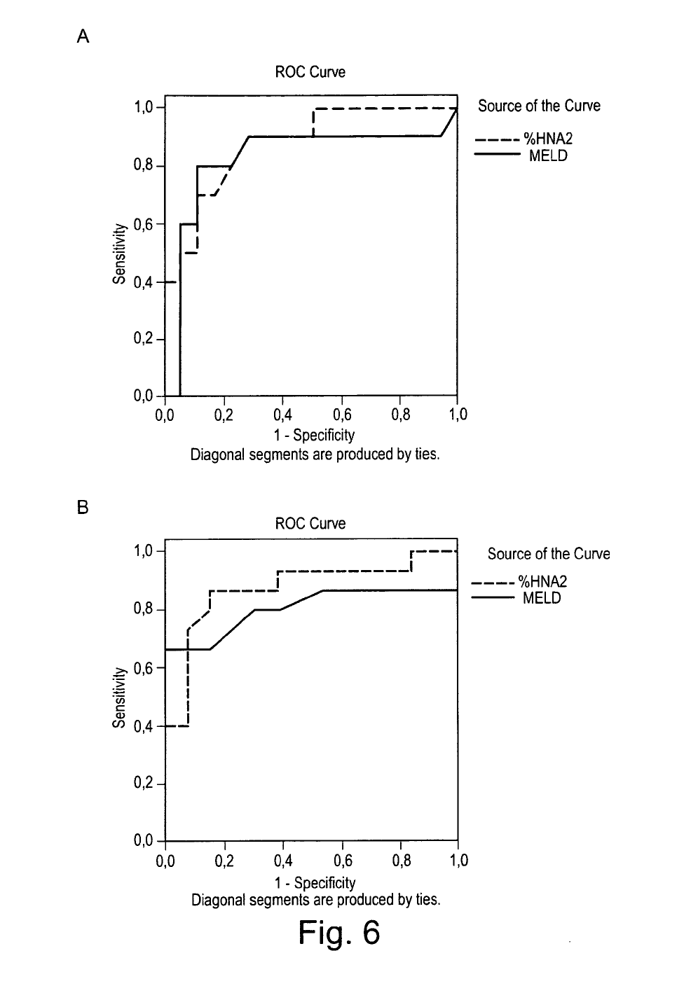

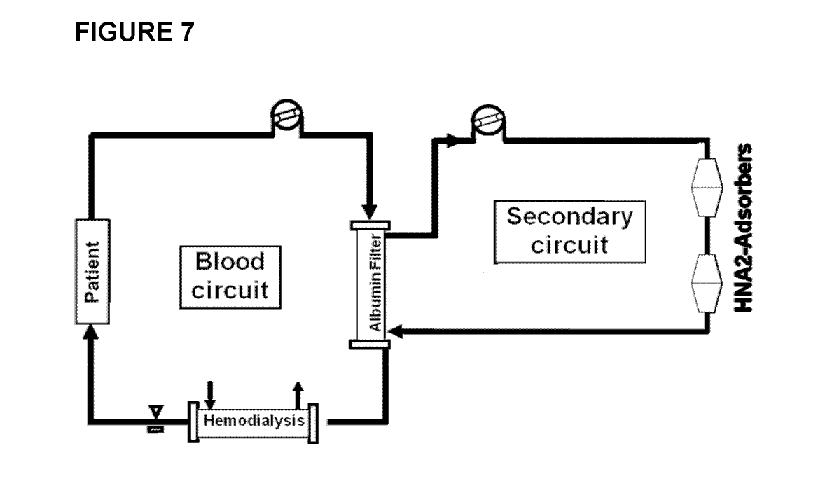

Septic patients showed increased albumin oxidation similar to cirrhotic patients and decreased OS binding. However, no significant correlations of oxidative modification and binding data were found for this group. The different behavior of septic and liver patients concerning correlations of IC₅₀ values is pointed out in FIG. 4. On the other hand, HNA2 reveals good correlation not only to bilirubin and INR but also to CRP in liver patients. These data suggest that albumin oxidation is closer related to inflammation than DS binding. In addition, factors resulting from impaired liver function like high bilirubin have a major impact on albumin's binding properties especially in ACLF patients. The possibility of allosteric effects of ligands leading to changes in binding of other ligands has been reported (Otagiri, M. (2005) Drug Metab. Pharmacokinet. 20, 309-323). It should be noted that the high content of the antioxidant bilirubin and the close spatial vicinity of cysteine 34 to the bilirubin binding sites for both high affinity binding and for covalent binding (lysine 190, FIG. 1) cannot protect cysteine 34 from oxidation (Adachi, Y. et al. (1996) Int. Hepatol. Commun. 5, 282-288). In analogy, it has been reported that binding of fatty acids to albumin enhances the susceptibility of cysteine 34 to oxidation (Gryzunov, Y. A. et al. (2003) Arch. Biochem. Biophys. 413, 53-66).

Bilirubin can bind to albumin at various sites (Vitek, L. et al. (2009) Curr. Pharm. Des. 15, 2869-2883). The primary site of high-affinity binding is still controversial (Zunszain, P. A. et al. (2008) J. Mol. Biol. 381, 394-406). While drug binding site I is generally believed to represent the major high-affinity binding site for bilirubin, evidence is accumulating that other sites are important as well. The spatial vicinity of cysteine 34 and the putative high-affinity bilirubin binding site (site I) as well as the recently described binding site and covalently bound bilirubin at lysine 190 (FIG. 1) may explain the previously reported influence of the redox state of cysteine 34 on bilirubin binding (Adachi, Y. et al. (1996), supra). Likewise, our finding that DS binding is more closely related to bilirubin than to HNA2 is consistent with the closer vicinity of site II to bilirubin binding sites than to cysteine 34 (FIG. 1).

Further evidence for impaired albumin function in advanced liver failure was recently obtained in a study of 20 patients with ACLF (Jalan, R. et al. (2009) Hepatology 50, 555-564). These authors demonstrated a reduced affinity of albumin fatty acid binding sites using electron paramagnetic resonance spectroscopy which was paralleled by a relative increase in ischemia modified albumin indicated by a reduced cobalt binding capacity.

The reduced albumin binding capacity in advanced liver failure and in sepsis has important clinical implications. Higher fractions of unbound ligands may increase their toxicity thus deteriorating the clinical course. Impaired binding, transport and delivery of drugs (e.g. antibiotics) may hinder specific treatment. Finally, the interaction of drugs and/or toxins with endogenous substances accumulating in liver failure may play an important pathogenetic role as they compete for a reduced number of functional binding sites on the damaged albumin molecules. For example, unbound endotoxin, which is normally bound to albumin in a 10:1 molecular ratio (Jurgens, G. et al. (2002) J. Endotoxin Res. 8, 115-126), could negatively influence innate immune function in liver disease. (Mookerjee, R. P. et al. (2007) Hepatology 46, 831-840). This negative effect can be reversed ex vivo by addition of albumin (Stadlbauer, V. et al. (2009) Am. J. Physiol. Gastrointest. Liver Physiol. 296, G15-G22).

It should be noted that the fraction of irreversibly oxidized albumin (HNA2) observed in ACLF is the highest of all patient groups studied so far (Oettl, K. and Marsche, G. (2010) Meth. Enzymol. 474, 181-195). Apparently, this finding has an important implication for recent concepts of artificial liver support. Cell-free extracorporeal liver support systems such as MARS or Prometheus have been shown to provide elimination of bilirubin, bile acids and other putative toxins accumulating in liver failure.

However, according to preliminary reports of two large randomized controlled trials, both systems failed to demonstrate a significant survival benefit in acute-on-chronic liver failure (Banares, R. et al. (2010) J. Hepatol. 52, 5459-5460; Rifai, K. et al. (2010) J. Hepatol. 52, S3). Although site II specific albumin binding capacity could be partly restored by MARS (Klammt, S. et al. (2008) Liver Transpl. 14, 1333-1339), it should be pointed out that the observed irreversible damage of circulating albumin could not be reversed by extracorporeal liver support (Jalan, R. et al. (2009), supra; Oettl, K. et al. (2009) Ther. Apher. Dial. 13, 431-436). Both MARS® and Prometheus® are designed to regenerate circulating albumin, but this is obviously hampered by irreversible damage of a large fraction of circulating albumin due to oxidative damage (as reflected by the high content of irreversibly oxidized HNA2) and/or other mechanisms such as the covalent binding of bilirubin to albumin (delta-bilirubin) (Weiss, J. S. et al. (1983) N. Engl. J. Med. 309, 147-150; Gautam, A. et al. (1984) J. Clin. Invest. 73, 873-877; Jansen, P. L. et al. (1986) J. Hepatol. 2, 485-494). Therefore, future liver support systems should conceptually aim at removal of irreversibly damaged albumin fractions and their replacement, rather than at mere ‘regeneration’ of albumin. It is hypothesized that partial albumin exchange, e.g. by plasmapheresis or selective plasma filtration, may be a more promising way to restore albumin function and thus improve multiorgan dysfunction and survival.

Finally, analysis of the prognostic value of HNA2 with respect to short-term survival revealed superior diagnostic accuracy as compared to MELD, the current standard for assessment of prognosis in chronic liver failure. However, the diagnostic accuracy of HNA2 has to be validated in larger cohorts of patients. If its prognostic value is confirmed in a large validation cohort, the development of alternate methods (other than HPLC) for measuring HNA2 at higher throughput is conceived.

Based on the above findings it can be concluded that impaired site II specific albumin binding capacity observed in chronic liver failure is mainly related to the severity of liver dysfunction. Concurrent oxidative stress enhances the loss of albumin function both in chronic liver failure and in sepsis. Irreversible oxidation of albumin is associated with prognosis and may be exploited as a new biomarker for liver dysfunction in chronic liver failure.

The present invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by embodiments and optional features, modifications and variations of the inventions embodied therein may be resorted to by those skilled in the art, and that such modifications and variations are considered to be within the scope of this invention.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

1. A method for diagnosing in a subject a medical condition associated with oxidative stress, the method comprising:
(a) determining in a test sample derived from the subject the redox state of albumin at the cysteine residue at sequence position 34 (cysteine 34);
(b) quantifying the level of non-mercaptalbumin-2 present in the test sample, nonmercaptalbumin-2 representing the albumin fraction being irreversibly oxidized at cysteine 34; and
(c) comparing the result obtained in step (b) to a control level, wherein an elevated level of non-mercaptalbumin-2 in the test sample derived from the subject as compared to the control level is indicative of the presence and/or prognosis of the medical condition associated with oxidative stress.

2. The method of claim 1, wherein the medical condition associated with oxidative stress is selected from the group consisting of sepsis, renal diseases, liver diseases, cardiovascular diseases, neurodegenerative diseases, rheumatologic diseases, premalignant and malignant diseases, and aging.

3. The method of claim 1, wherein the test sample is a blood sample, and in particular a plasma sample.

4. The method of claim 1, wherein the subject is a human subject.

5. The method of claim 1, further comprising:
(d) determining in the test sample the binding capacity of albumin for aromatic carboxylic compounds; and
(e) comparing the result obtained in step (d) to a control, wherein a decreased binding capacity of albumin in the test sample as compared to the control is indicative of the presence and/or prognosis of the medical condition associated with oxidative stress.

6. The method of claim 1, wherein the level of non-mercaptalbumin-2 present in the test sample is quantified by means of a technique being selected from the group consisting of a chromatographic technique and a mass spectrometric technique.

7. The method of claim 1, wherein the level of non-mercaptalbumin-2 present in the test sample is quantified by means of an antibody-based technique.

8. The method of claim 7, wherein the antibody molecule employed exhibits binding specificity for non-mercaptalbumin-2 comprising cysteine 34 in irreversibly oxidized form and binds to the target with an affinity of less than 1 μM, and in particular of less than 100 nM.

9. The method of claim 8, wherein the antibody molecule employed exhibits binding specificity for an epitope comprising cysteine 34 in irreversibly oxidized form.

10. An antibody molecule exhibiting binding specificity for non-mercaptalbumin-2 comprising:
cysteine 34 in irreversibly oxidized form and binding to the target with an affinity of less than 1 μM, and in particular of less than 100 nM.

11. The antibody molecule of claim 10, exhibiting binding specificity for an epitope comprising cysteine 34 in irreversibly oxidized form.

12. The method of claim 1, wherein quantifying the level of non-mercaptalbumin-2 present in the test sample is derived from the level of non-mercaptalbumin-2 that is elevated as compared to a control, wherein such an elevated level of non-mercaptalbumin-2 in the test sample is indicative of the presence of the medical condition associated with oxidative stress; and further comprising:
(d) restoring a control level of albumin by performing either one or both selected from the group consisting of specifically removing excess non-mercaptalbumin-2 and exogenously adding albumin.

13. The method of claim 12, wherein the method is performed in vitro.

14. A method of use of non-mercaptalbumin-2 as a biomarker for diagnosing in a subject a medical condition associated with oxidative stress.

15. The method of claim 14, wherein the medical condition associated with oxidative stress is selected from the group consisting of sepsis, renal diseases, liver diseases, cardiovascular diseases, neurodegenerative diseases, rheumatologic diseases, premalignant and malignant diseases, and aging.
